(19) United States
(12) Patent Application Publication
(10) Pub. No.: US 2021/0080477 A1
(43) Pub. Date: Mar. 18, 2021

(54) FULL-AUTOMATIC CHEMILUMINESCENCE IMMUNOASSAY ANALYZER

(71) Applicant: Jiangsu Zecen Biotechnology Co., Ltd., Jiangyin (CN)

(72) Inventors: Ming JIN, Jiangyin (CN); Yan GAO, Jiangyin (CN); Zhenshi LIU, Jiangyin (CN); Junjie WANG, Jiangyin (CN)

(73) Assignee: Jiangsu Zecen Biotechnology Co., Ltd.

(21) Appl. No.: 17/086,491

(22) Filed: Nov. 2, 2020

Related U.S. Application Data
(63) Continuation of application No. PCT/CN2020/117130, filed on Sep. 23, 2020.

(30) Foreign Application Priority Data
Dec. 19, 2019 (CN) 201911319725.6

(51) Int. Cl. G01N 35/00; G01N 35/10; G01N 21/76

(52) U.S. Cl. CPC G01N 35/0099; G01N 35/10; G01N 2035/00891; G01N 2035/00356; G01N 2035/00534; G01N 21/763

(57) ABSTRACT

The present invention discloses a full-automatic chemiluminescence immunoassay analyzer, which relates to the field of luminescence analysis equipment. The analyzer of the present invention includes a reaction tube module, a sample tube module, a kit module, a three-dimensional mechanical arm module, a test tube fetching gripper apparatus, a blending structure, an incubator, a washing apparatus, an electric control part and a PC control end. The three-dimensional mechanical arm module includes an X-axis mechanical arm, a Y-axis mechanical arm and a Z-axis mechanical arm, and the bottom of the Z-axis mechanical arm is provided with a sampling needle. The present invention utilizes the test tube fetching gripper apparatus to substitute the original tube fetching and placing apparatus, thereby reducing the cost and improving the moving stability of reaction tubes.

### TECHNICAL FIELD

The present invention relates to the field of luminescence analysis equipment, and particularly relates to a full-automatic chemiluminescence immunoassay analyzer.

### BACKGROUND

Chemiluminescence immunoassay is a clinical laboratory technique for detecting antigens or antibodies in human body fluid. Because of its high sensitivity, good selectivity and other advantages, the chemiluminescence immunoassay has been widely used. At present, it has changed to a common means in clinical immunological test from the theoretical research in the laboratory. Correspondingly, the development of various chemiluminescence immunoassay instruments has become more and more mature. Based on the antigen-antibody reaction principle, the chemiluminescence amplification technology marks enzymes or other non-radioactive markers on antigens or antibodies, which then react with known antigens or antibodies. The marked enzymes make a reaction substrate emit light, which is measured by a photon counter to obtain the luminescence count per second of a tested sample, and the luminescence count can be converted into a concentration value of the sample according to a built-in standard curve. By using the technology, the antigen-antibody reaction time is shortened, the specificity and the sensitivity are improved, and the full automation of the whole reaction becomes possible.

Our company developed an automatic chemiluminescence immunoassay analyzer previously. The specific content can be seen in the invention patent with the publication No. CN103743916A and the title of a full-automatic chemiluminescence immunoassay analyzer. On this basis, our company made further optimization, so that the full-automatic chemiluminescence immunoassay analyzer is more stable, higher in test throughput, low in cost, simple in installation and good in stability, and can make samples, reagents and magnetic particles react more sufficiently.

### SUMMARY

The present invention provides a full-automatic chemiluminescence immunoassay analyzer, which solves the problem of the further optimization of the existing analyzer.

To solve the above technical problems, the present invention adopts the following technical solutions:

The full-automatic chemiluminescence immunoassay analyzer includes a reaction tube module, a sample tube module, a kit module, a three-dimensional mechanical arm module, a test tube fetching gripper apparatus, a blending structure, an incubator, a washing apparatus, an electric control part and a PC control end. The three-dimensional mechanical arm module includes an X-axis mechanical arm, a Y-axis mechanical arm and a Z-axis mechanical arm, and the bottom of the Z-axis mechanical arm is provided with a sampling needle. The test tube fetching gripper apparatus is installed beside the reaction tube module. The blending structure includes a blending motor; a power output end of the blending motor is provided with a first blending wheel; the first blending wheel is provided with a second blending wheel; the first blending wheel is connected with the second blending wheel through a bearing; the second blending wheel is a hollow structure; one side of the second blending wheel is provided with an “inverted U-shaped” blending separation blade; the outer wall of the second blending wheel is fixedly connected with a blending shift lever; and the blending shift lever stretches into the blending separation blade.

Preferably, the three-dimensional mechanical arm module includes the X-axis mechanical arm, the Y-axis mechanical arm and the Z-axis mechanical arm. The X-axis mechanical arm includes an installation platform. The installation platform is provided with a wide linear guide rail. The Y-axis mechanical arm is slidably installed on the wide linear guide rail through an X-axis bottom plate. The Z-axis mechanical arm is movably installed on the Y-axis mechanical arm through a Y-axis transmission belt. The installation platform is provided with an X-axis driving wheel and an X-axis driven wheel along an X axis. The X-axis driving wheel is connected with the power output end of the X-axis motor. The X-axis driving wheel and the X-axis driven wheel are sleeved with an X-axis belt. The X-axis bottom plate is fixedly connected with the X-axis belt.

Preferably, the inner wall of the hollow structure of the second blending wheel is provided with an anti-skid layer.

Preferably, the Y-axis mechanical arm and the Z-axis mechanical arm of the three-dimensional mechanical arm module are covered with a dust-proof hood.

Preferably, the test tube fetching gripper apparatus is installed in an installation casing.

Preferably, the reaction tube module, the sample tube module, the kit module, the three-dimensional mechanical arm module, the test tube fetching gripper apparatus, the blending structure, the incubator and the washing apparatus are installed in a transparent stander.

By adopting the above technical solution, the present invention utilizes the test tube fetching gripper apparatus to substitute the original test tube fetching and placing apparatus, thereby reducing the cost, and improving the moving stability of the reaction tube. A previously used syringe-pump tube moving structure fetches and stores the reaction tube in a way similar to a pipette, but occasionally has the situation that the reaction tube cannot be taken down. The improved test tube fetching gripper apparatus has two gripper sheets to hold the reaction tube, and the gripper sheets are unfolded to release the reaction tube, so that the cost is reduced, the installation is simple, and the stability is good. The present invention adds the blending structure on the basis of the original mechanical structure, so that the samples, reagents and magnetic particles can react more sufficiently, and the pollutant carrying rate can be reduced.

### BRIEF DESCRIPTION OF THE DRAWINGS

FIG. 1 is a structural schematic diagram of the present invention.

FIG. 2 is a structural schematic diagram of a three-dimensional mechanical arm module in a direction of the present invention.

FIG. 3 is a structural schematic diagram of the three-dimensional mechanical arm module in another direction of the present invention.

FIG. 4 is a structural schematic diagram of a blending structure of the present invention.

FIG. 5 is a main section view of the blending structure of the present invention.

In the drawings, 1-three-dimensional mechanical arm module; 1-1-installation platform; 1-2-wide linear guide rail; 1-3-X-axis bottom plate; 1-4-Z-axis mechanical arm; 1-5-Y-axis transmission belt; 1-6-X-axis driving wheel; 1-7-X-axis driven wheel; 1-8-X-axis motor; 1-9-X-axis belt; 1-10-dust-proof hood; 2-test tube fetching gripper apparatus; 3-blending structure; 3-1-blending motor; 3-2-first blending wheel; 3-3-second blending wheel; 3-4-bearing; 3-5-blending separation blade; 3-6-blending shift lever; 4-sample tube module; 5-kit module; 6-reaction tube module; 7-incubator; 8-sampling needle; 9-washing apparatus; 10-PC control end; 11-transparent stander.

### DETAILED DESCRIPTION

Specific embodiments of the present invention are further described below in combination with the accompanying drawings. It should be noted herein that the description of these embodiments is used to help to understand the present invention, but does not constitute a limitation to the present invention. Furthermore, the technical features involved in various embodiments of the present invention described below can be combined if there is no contradiction.

In the description of the present invention, it should be noted that the terms “center”, “longitudinal”, “transverse”, “length”, “width”, “thickness”, “upper”, “lower”, “front”, “rear”, “left”, “right”, “vertical”, “horizontal”, “top”, “bottom”, “inside”, “outside”, “clockwise”, “counterclockwise”, “axial”, “radial”, “peripheral” and the like indicating the orientation or positional relationship are based on the orientation or positional relationship shown in the drawings, and are only used for convenience in describing the present invention and simplifying the description, rather than indicating or implying that specific devices or elements must have a specific orientation and must be constructed and operated in a specific orientation. Therefore, the terms shall not be understood as limitations to the present invention.

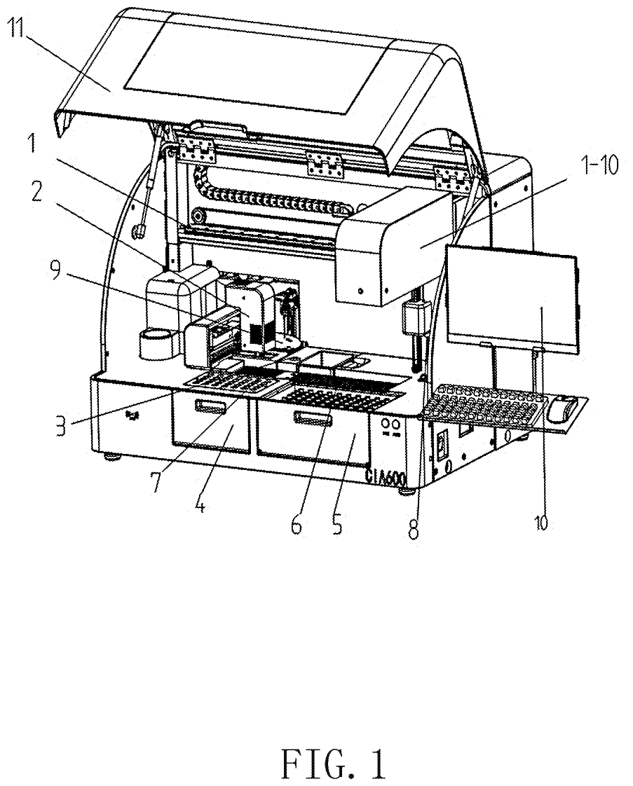

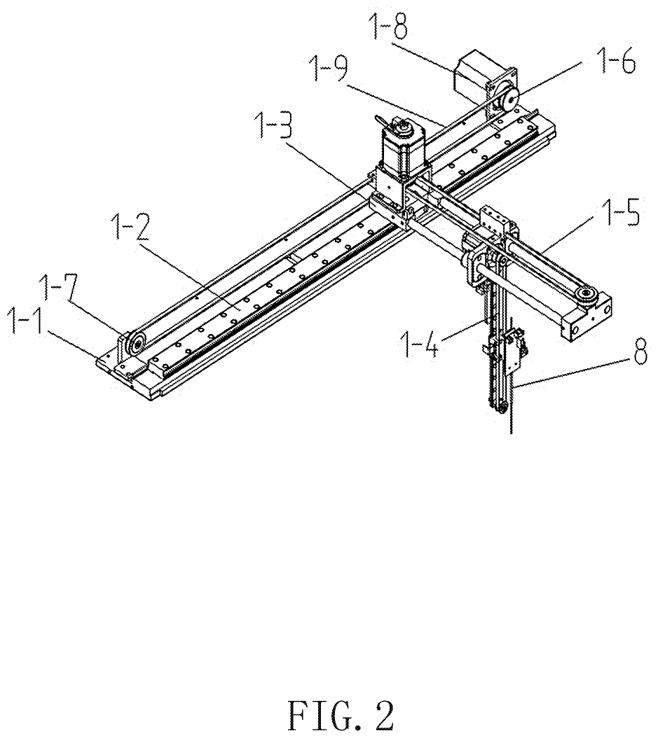

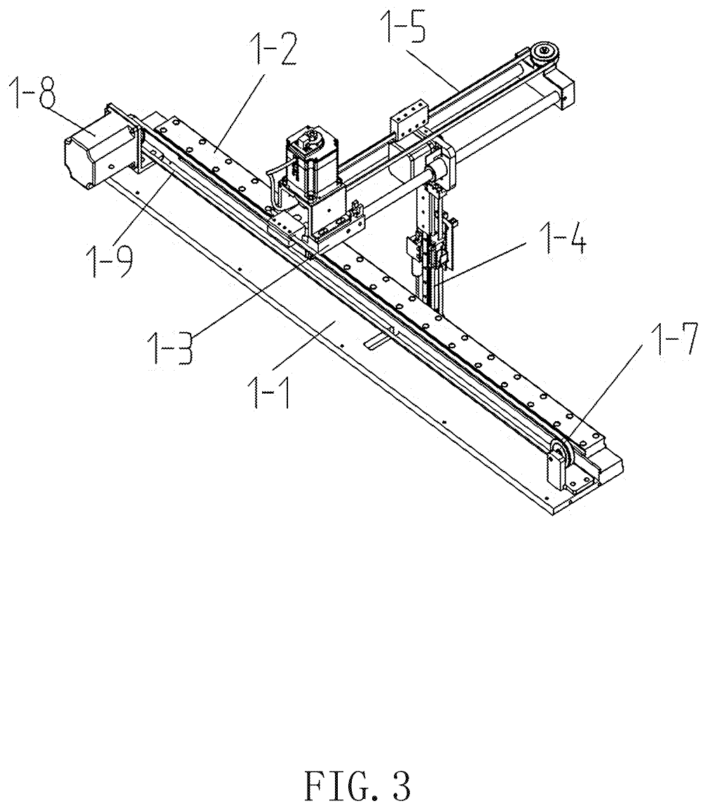

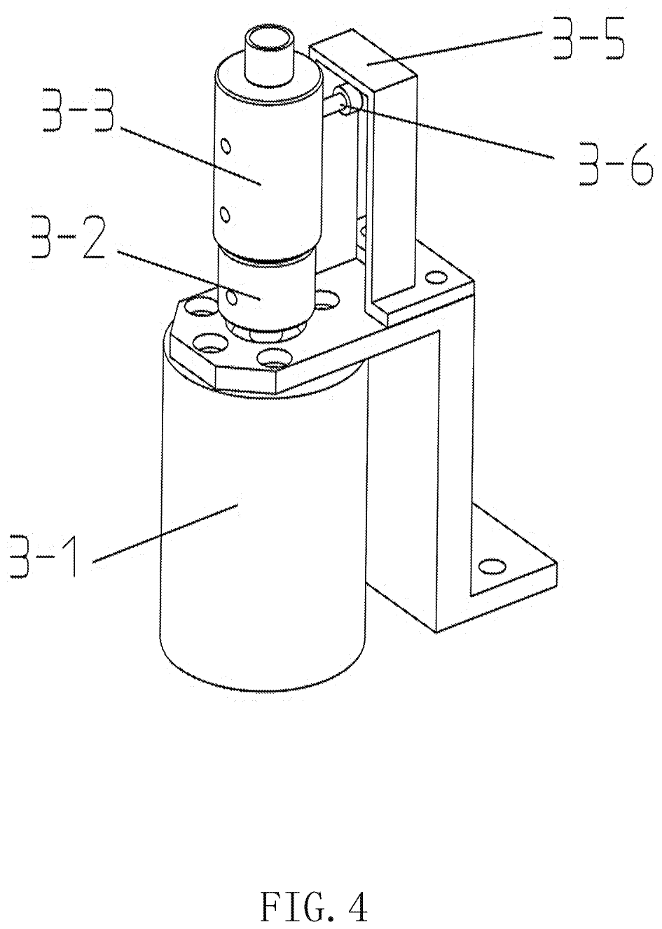

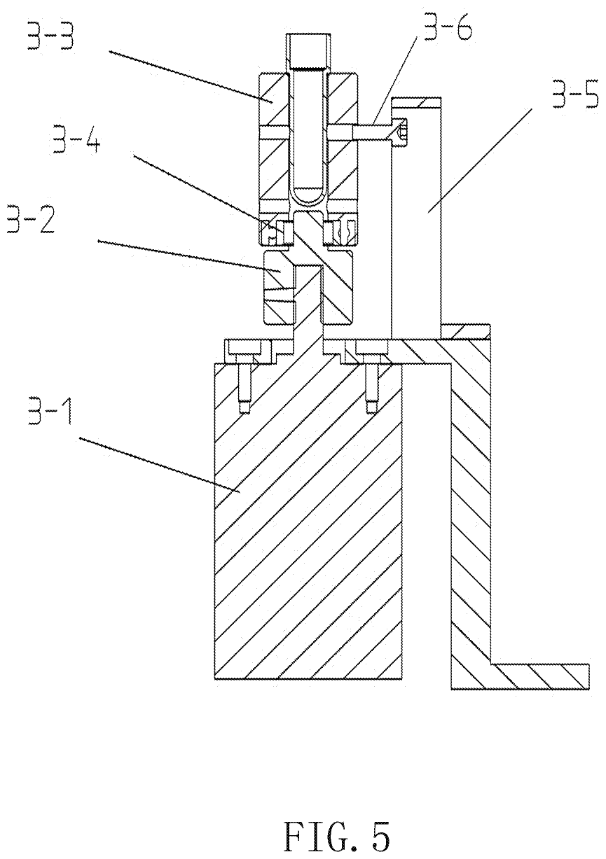

As shown in FIGS. 1-5, a full-automatic chemiluminescence immunoassay analyzer includes a reaction tube module 6, a sample tube module 4, a kit module 5, a three-dimensional mechanical arm module 1, a test tube fetching gripper apparatus 2, a blending structure 3, an incubator 7, a washing apparatus 9, an electric control part and a PC control end 10. The three-dimensional mechanical arm module 1 includes an X-axis mechanical arm, a Y-axis mechanical arm and a Z-axis mechanical arm 1-4, and the bottom of the Z-axis mechanical arm 1-4 is provided with a sampling needle 8 which can be driven by the three-dimensional mechanical arm module 1 to add a sample, a reagent and magnetic particles in the sample tube module 4 and the kit module 5 respectively into reaction tubes of the reaction tube module 6. The test tube fetching gripper apparatus 2 is installed beside the reaction tube module 6, and the specific structure of the test tube fetching gripper apparatus 2 can be referred to the patent invention of our company with the publication No. CN207373198U and the title of a test tube gripper apparatus. The blending structure 3 includes a blending motor 3-1; a power output end of the blending motor 3-1 is provided with a first blending wheel 3-2; the first blending wheel 3-2 is provided with a second blending wheel 3-3; the first blending wheel 3-2 is connected with the second blending wheel 3-3 through a bearing 3-4; and the second blending wheel 3-3 is a hollow structure, so that the reaction tubes can be placed into the second blending wheel 3-3 for uniform mixing. One side of the second blending wheel 3-3 is provided with an “inverted U-shaped” blending separation blade 3-5, the outer wall of the second blending wheel 3-3 is fixedly provided with a blending shift lever 3-6, and the blending shift lever 3-6 stretches into the blending separation blade 3-5.

When in use, the sample tube module 4, the kit module 5 and the reaction tube module 6 are respectively used for storing the samples, the reagents and the reaction tubes. After the experiment is started, the three-dimensional mechanical arm module 1 drives the sampling needle 8 to add the sample, the reagent and the magnetic particle sample in the sample tube module 4 and the kit module 5 into the reaction tubes of the reaction tube module 6. The test tube fetching gripper apparatus 2 may fetch the reaction tubes filled with the liquid into the blending structure 3 for uniform mixing. The blending motor 3-1 swivels to drive the first blending wheel 3-2 fixed on the shaft end of the motor to swivel together. The first blending wheel 3-2 further drives the second blending wheel 3-3 to swivel together with the reaction tube in the second blending wheel 3-3. The second blending wheel 3-3 is connected with the first blending wheel 3-2 through the bearing. When the second blending wheel 3-3 swivels, the blending shift lever 3-6 on the second blending wheel 3-3 may be blocked by the blending separation blade 3-5, and at this time, the second blending wheel 3-3 may form vibration in the blending separation blade 3-5, so that the sample, the reagent and the magnetic particles in the reaction tube are uniformly and fully mixed. After the uniform mixing, the test tube fetching gripper apparatus 2 places the reaction tube into the incubator 7 for reaction and incubation. After the reaction, the test tube fetching gripper apparatus 2 grabs the reaction tube into the washing apparatus 9 to be washed and detected, and the detection result may be displayed on the PC control end 10.

The present invention utilizes the test tube fetching gripper apparatus 2 to substitute the original tube fetching and placing apparatus, thereby reducing the cost and improving the moving stability of the reaction tube. A previously used syringe-pump tube moving structure fetches and stores the reaction tube in a way similar to a pipette, but occasionally has the situation that the reaction tube cannot be taken down. The improved test tube fetching gripper apparatus 2 has two gripper sheets to hold the reaction tube, and the gripper sheets are unfolded to release the reaction tube, so that the cost is reduced, the installation is simple, and the stability is good. The present invention adds the blending structure 3 on the basis of the original mechanical structure, so that the samples, reagents and magnetic particles can react more sufficiently, and the pollutant carrying rate can be reduced.

Specifically, the three-dimensional mechanical arm module 1 includes the X-axis mechanical arm, the Y-axis mechanical arm and the Z-axis mechanical arm 1-4. The X-axis mechanical arm includes an installation platform 1-1. The installation platform 1-1 is provided with a wide linear guide rail 1-2. The Y-axis mechanical arm is slidably installed on the wide linear guide rail 1-2 through an X-axis bottom plate 1-3. The Z-axis mechanical arm 1-4 is movably installed on the Y-axis mechanical arm through a Y-axis transmission belt 1-5. The installation platform 1-1 is provided with an X-axis driving wheel 1-6 and an X-axis driven wheel 1-7 along the X axis. The X-axis driving wheel 1-6 is connected with a power output end of the X-axis motor 1-8. The X-axis driving wheel 1-6 and the X-axis driven wheel 1-7 are sleeved with an X-axis belt 1-9. The X-axis bottom plate 1-3 is fixedly connected with the X-axis belt 1-9. Under the driving of the X-axis motor 1-8, the X-axis belt 1-9 can move between the X-axis driving wheel 1-6 and the X-axis driven wheel 1-7 along the X axis to drive the Y-axis mechanical arm on the X-axis bottom plate 1-3 to move in the X-axis direction. Two cylindrical guide rails of the original X-axis mechanical arm are optimized into the single wide linear guide rail 1-2, so that the installation requirement is low; the installation requirement of the two cylindrical guide rails of the original X-axis mechanical arm is high; the two cylindrical arms are required to be parallel to each other; otherwise, the smooth movement of the mechanical arm may be affected, easily causing the seizure phenomenon. The optimized X-axis mechanical arm is higher in stability, lower in noise and more stable and efficient in operation. The present invention optimizes the two cylindrical guide rails of the original X-axis mechanical into the single wide linear guide rail 1-2, so that the installation requirement is low. The installation requirement of the two cylindrical guide rails of the original X-axis mechanical arm is high, and the two cylindrical guide rails are required to be parallel to each other; otherwise, the smooth movement of the mechanical arm may be affected, easily causing the seizure phenomenon. The optimized X-axis mechanical arm is higher in stability, lower in noise and more stable and efficient in operation.

In order to ensure the vibration stability of the reaction tube added into the second blending wheel 3-3, the inner wall of the hollow structure of the second blending wheel 3-3 is provided with an anti-skid layer which may be made of rubber and other materials, so that the effective anti-skid effect can be guaranteed, and the reaction tube can be prevented from throwing away in the vibration process.

In order to prolong the service life and improve the attractiveness of the prevent invention, the Y-axis mechanical arm and the Z-axis mechanical arm 1-4 of the three-dimensional mechanical arm module 1 are covered with a dust-proof hood 1-10.

In order to prevent the test tube fetching gripper apparatus 2 from being seized or polluted during the application, the test tube fetching gripper apparatus 2 is installed in an installation casing, so that not only can the service life of the test tube fetching gripper apparatus 2 be prolonged, but also the attractiveness can be improved.

In order to improve the use monitoring performance and stability of the present invention, the reaction tube module 6, the sample tube module 4, the kit module 5, the three-dimensional mechanical arm module 1, the test tube fetching gripper apparatus 2, the blending structure 3, the incubator 7 and the washing apparatus 9 are installed in a transparent stander 11, so that the internal working condition can be monitored by the working personnel in the reaction process, and the service life of the present invention can be prolonged; and moreover, the transparent stander 11 is provided with a window, which is convenient to open for operation.

The above only describes the preferred embodiments of the present invention, and is not intended to limit the present invention in any form. Although the present invention has been disclosed as above in preferred embodiments, it is not limited thereby. Those skilled in the art can make changes or modifications to form equivalent embodiments on the basis of the technical content disclosed above without departing from the scope of the technical solution of the present invention, and any simple changes, equivalent alterations and modifications made on the basis of the technical essence of the present invention without departing from the content of the technical solutions of the present invention shall be still within the scope of the technical solutions of the present invention.

1. A full-automatic chemiluminescence immunoassay analyzer, comprising a reaction tube module (6), a sample tube module (4), a kit module (5), a three-dimensional mechanical arm module (1), a test tube fetching gripper apparatus (2), a blending structure (3), an incubator (7), a washing apparatus (9), an electric control part and a PC control end (10), wherein the three-dimensional mechanical arm module (1) comprises an X-axis mechanical arm, a Y-axis mechanical arm and a Z-axis mechanical arm (1-4), and the bottom of the Z-axis mechanical arm (1-4) is provided with a sampling needle (8); the test tube fetching gripper apparatus (2) is installed beside the reaction tube module (6); the blending structure (3) comprises a blending motor (3-1); a power output end of the blending motor (3-1) is provided with a first blending wheel (3-2); the first blending wheel (3-2) is provided with a second blending wheel (3-3); the first blending wheel (3-2) is connected with the second blending wheel (3-3) through a bearing (3-4); the second blending wheel (3-3) is a hollow structure; one side of the second blending wheel (3-3) is provided with an “inverted U-shaped” blending separation blade (3-5); the outer wall of the second blending wheel (3-3) is fixedly connected with a blending shift lever (3-6); and the blending shift lever (3-6) stretches into the blending separation blade (3-5).

2. The full-automatic chemiluminescence immunoassay analyzer according to claim 1, wherein the three-dimensional mechanical arm module (1) comprises the X-axis mechanical arm, the Y-axis mechanical arm and the Z-axis mechanical arm (1-4); the X-axis mechanical arm comprises an installation platform (1-1); the installation platform (1-1) is provided with a wide linear guide rail (1-2); the Y-axis mechanical arm is slidably installed on the wide linear guide rail (1-2) through an X-axis bottom plate (1-3); the Z-axis mechanical arm (1-4) is movably installed on the Y-axis mechanical arm through a Y-axis transmission belt (1-5); the installation platform (1-1) is provided with an X-axis driving wheel (1-6) and an X-axis driven wheel (1-7) along an X axis; the X-axis driving wheel (1-6) is connected with the power output end of the X-axis motor (1-8); the X-axis driving wheel (1-6) and the X-axis driven wheel (1-7) are sleeved with an X-axis belt (1-9); and the X-axis bottom plate (1-3) is fixedly connected with the X-axis belt (1-9).

3. The full-automatic chemiluminescence immunoassay analyzer according to claim 1, wherein the inner wall of the hollow structure of the second blending wheel (3-3) is provided with an anti-skid layer.

4. The full-automatic chemiluminescence immunoassay analyzer according to claim 1, wherein the Y-axis mechanical arm and the Z-axis mechanical arm (1-4) of the three-dimensional mechanical arm module (1) are covered with a dust-proof hood (1-10).

5. The full-automatic chemiluminescence immunoassay analyzer according to claim 1, wherein the test tube fetching gripper apparatus (2) is installed in an installation casing.

6. The full-automatic chemiluminescence immunoassay analyzer according to claim 1, wherein the reaction tube module (6), the sample tube module (4), the kit module (5), the three-dimensional mechanical arm module (1), the test tube fetching gripper apparatus (2), the blending structure (3), the incubator (7) and the washing apparatus (9) are installed in a transparent stander (11).
